(19) United States
(12) Patent Application Publication
(10) Pub. No.: US 2013/0344083 A1
(43) Pub. Date: Dec. 26, 2013

(54) MODULATION OF HISTONE H2B MONOUBIQUITINATION AND TREATMENT OF CANCER

(71) Applicant: Xu, C. Wilson, (unknown)

(72) Inventors: C. Wilson Xu, Pasadena, CA (US); Yasuyo Urasaki, Las Vegas, NV (US)

(21) Appl. No.: 13/829,075

(22) Filed: Mar. 14, 2013

Related U.S. Application Data
(60) Provisional application No. 61/638,018, filed on Apr. 25, 2012.

(51) Int. Cl. G01N 33/68

(52) U.S. Cl. CPC G01N 33/6893
USPC 424/158.1; 514/44 R; 514/44 A; 514/689; 514/23; 514/557; 514/406; 514/563; 514/178; 514/256; 514/635; 514/476; 514/738; 424/673; 424/644; 424/623; 530/389.1; 536/23.1; 536/24.5; 435/7.9

(57) ABSTRACT

Provided are methods and compositions for treatment of cancer. In particular, these methods and compositions may include an inhibitor of a deubiquitinating enzyme. In certain aspects, these methods and compositions may include a modulator of glucose metabolism. Also provided are methods of assaying the glucose content of cells and tissues using detection of uH2B.

### STATEMENT REGARDING FEDERALLY SPONSORED RESEARCH

This invention was made, at least in part, with government support under grant number W81XWH-10-1-0046 awarded by the Department of Defense. The U.S. government has certain rights in the invention.

### BACKGROUND OF THE INVENTION

Cancer cells exhibit aberrant glucose metabolism characterized by aerobic glycolysis, a phenomenon also known as the Warburg effect (Warburg 0 (1956) On the origin of cancer cells, Science 123: 309-314; Warburg 0, Wind F, Negelein E (1927) The Metabolism of Tumors in the Body, J Gen Physiol 8: 519-530). This metabolic reprogramming is thought to play an important role in supplying proliferating tumors with necessary building blocks for biomass production. Evidence also indicates that oncogenes and tumor suppressors play opposing roles in regulating glucose metabolism. (Vander Heiden M G, Cantley L C, Thompson C B (2009) Understanding the Warburg effect: the metabolic requirements of cell proliferation. Science 324: 1029-1033).

Using mono-ubiquitination of histone H2B at K123 (uH2B) in yeast as a model, it has been shown that glucose induces uH2B through glycolysis, revealing a paradigm of nutritional regulation of histone modifications. (Dong L, Xu C W (2004) Carbohydrates induce mono-ubiquitination of H2B in yeast. J Biol Chem 279: 1577-1580). It has been also demonstrated that glycolysis is also required for mono-ubquitination of histone H2B at K120, the orthologous site of K123 of yeast histone H2B, in both human primary and tumor cells (Z. Gao, and C. W. Xu, Glucose metabolism induces mono-ubiquitination of histone H2B in mammalian cells, Biochem Biophys Res Commun 404 (2011) 428-33).

Analysis of gene expression profiles has identified USP22 as a death-from-cancer gene in patients with MPM and other solid tumors (Glinsky G V, Berezovska O, Glinskii A B (2005) Microarray analysis identifies a death-from-cancer signature predicting therapy failure in patients with multiple types of cancer. J Clin Invest 115: 1503-1521). USP22 catalyzes the ubiquitin removal of mono-ubiquitinated histone H2B at K120 in human cells (uH2B) (Zhao Y, Lang G, Ito S, Bonnet J, Metzger E, et al. (2008) A TFTC/STAGA module mediates histone H2A and H2B deubiquitination, coactivates nuclear receptors, and counteracts heterochromatin silencing, Mol Cell 29: 92-101; Zhang X Y, Varthi M, Sykes S M, Phillips C, Warzecha C, et al. (2008) The putative cancer stem cell marker USP22 is a subunit of the human SAGA complex required for activated transcription and cell-cycle progression, Mol Cell 29: 102-111; Zhang X Y, Pfeiffer H K, Thorne A W, McMahon S B (2008) USP22, an hSAGA subunit and potential cancer stem cell marker, reverses the polycomb-catalyzed ubiquitylation of histone H2A. Cell Cycle 7: 1522-1524). USP22 is part of the SAGA (Spt-Ada-Gcn5 Acetyltransferase) chromatin remodeling complex, which functions as a transcriptional co-activator (Lee K K, Workman J L (2007) Histone acetyltransferase complexes: one size doesn't fit all. Nat Rev Mol Cell Biol 8: 284-295). USP22-containing SAGA is required for the expression of c-Myc target genes in lung cancer cells and for androgen receptor-mediated transactivation. In addition to deubiquitinating histones, USP22 participates in telomere maintenance by deubiquitinating non-histone proteins, such as telomeric-repeat-binding factor 1 (TRF1) (Atanassov B S, Evrard Y A, Multani A S, Zhang Z, Tora L, et al. (2009) Gcn5 and SAGA regulate shelterin protein turnover and telomere maintenance. Mol Cell 35: 352-364) and far upstream element (FUSE)-binding protein 1 (FBP1) (Atanassov B S, Dent S Y (2011) USP22 regulates cell proliferation by deubiquitinating the transcriptional regulator FBP1. EMBO Rep 12: 924-930).

### BRIEF SUMMARY OF THE INVENTION

In one aspect, the present invention provides a method of treatment for cancer comprising administration to a subject in need of treatment a composition comprising a negative modulator of a deubiquitinating enzyme or a deubiquitinating enzyme cofactor

In another aspect, the present invention provides a method of treatment for cancer comprising administration to a subject in need of treatment a composition comprising a modulator of glucose metabolism.

In another aspect, the present invention provides a method of assaying the glucose content of a cell or tissue comprising detecting the level of uH2B in the cell or tissue.

In a further aspect, the present invention provides a composition comprising at least one negative modulator of a deubiquitinating enzyme or a deubiquitinating enzyme cofactor .

### BRIEF DESCRIPTION OF THE DRAWINGS

FIG. 1 demonstrates that glucose levels in tumors are lower than those of normal tissues of the same tissue sites. Five pairs of matched tumor and normal tissue specimens from Biochain (B) and Origene (0) were analyzed for glucose and protein content. The amount of glucose was normalized with the total protein concentration. Each sample was assayed in quadruplicate.

FIG. 2 demonstrates that uH2B is a semi-quantitative histone marker for glucose. Stationary phase (SP) yeast (Y117) was incubated with different amounts of glucose for 1 hr. Brain (U87), Breast (MCF7) and colon (HCT116) cancer cell lines were grown in complete media (10% FBS/DMEM) until 40-60% confluency and subsequently incubated with DMEM containing 10% dialyzed FBS with indicated amounts of glucose for 24 hrs. The glucose (Glc) concentrations used in the assays covered the physiological serum glucose levels (0.07-0.12%). uH2B levels in these cells corresponded to those of glucose semi-quantitatively. Half of the glucose-treated tumor cells were also formalin-fixed and paraffin-embedded for immunohistochemical staining of uH2B and H2B (FIG. 4). uH2B levels, detected by immunohistochemistry, were also proportional to those of glucose, further indicating that uH2B may be used as a chromatin marker for glucose.

FIGS. 3A-C demonstrate immunohistochemical staining of uH2B and H2B in tumor cells. The cells, from the same batches of the glucose-treated cells that were used in Western blotting analysis (FIG. 2), were formalin-fixed and paraffin-embedded for immunohistochemical staining of uH2B and H2B. These cells were subsequently counterstained with Hematoxylin. At least 1000 cells were examined for each sample. uH2B levels correlated with the amounts of glucose (Glc) of the media for culturing glioblastoma cells (U87, A), breast cancer (MCF7, B) and colon cancer (HTC116, C) cells. Scale bar=50 μM.

FIGS. 4A-C demonstrate that glucose-induced uH2B is significantly impaired in cancer cells compared to their adjacent stromal tissues. Human breast, colon and lung tumor specimens from surgery were immunohistochemically stained for uH2B and H2B and subsequently counter-stained with Hematoxylin. A. uH2B levels are inhibited in breast cancer cells in 36 out of 37 cases. Two representative cases are shown. Intense staining of uH2B was observed in normal myoepithelial and luminal epithelial cells of Duct 1 of a tumor specimen from breast cancer patient BC-D9. Although it was in the tumor specimen, Duct 1 had no cancer cells. uH2B was significantly reduced in luminal epithelial cancer cells, which was encircled with a dashed line in Duct 2. In contrast, uH2B remained high in normal luminal epithelial cells in Duct 2. Other cancer cells in BC-D9 tumor specimen were not separated with dashed lines for the purpose of clarity. Breast cancer cells from patient BC-01 also showed low uH2B staining, whereas adjacent normal cells maintained high uH2B staining. B. uH2B levels are drastically reduced in colon cancer cells in 35 out of 36 cases. Two representative cases are shown. C. uH2B levels are significantly inhibited in lung cancer cells in 35 out of 36 cases. Two representative cases are shown. The black arrow shows a piece of cigarette tar. N denotes normal cells or stromal tissues. C denotes cancer cells. Dashed lines demarcate cancer cells from their adjacent normal or stromal cells. Scale bar=50 μM.

FIGS. 5A-D demonstrate that, in vitro, expression of USP22 is increased by glucose deficiency, while glucose deficiency inhibits uH2B.

FIGS. 6A-C demonstrate that USP22 is over-expressed in cancer cells of breast, colon and lung cancers, whereas and uH2B is reduced or impaired in the same breast, colon, and lung cancer cells of patient tumor specimens.

### DETAILED DESCRIPTION OF THE INVENTION

Metabolic reprogramming is associated with tumorigenesis. In accordance with the present invention, it has been demonstrated that glucose levels are significantly lower in bulk tumor specimens than those in normal tissues of the same tissue origins. Mono-ubiquitinated histone H2B (uH2B) is demonstrated to be a semi-quantitative histone marker for glucose. Further, it has been discovered that loss of uH2B occurs specifically in cancer cells from a wide array of tumor specimens of breast, colon, lung and an additional twenty-three (23) anatomic sites. In contrast, uH2B levels remain high in stromal tissues or non-cancerous cells in tumor specimens. Taken together, these data indicate that glucose deficiency and loss of uH2B are properties of cancer cells in vivo, which may represent important regulatory mechanisms of tumorigenesis.

While not intending to be bound by any theory of operation, glucose deprivation in cancer cells may be a molecular basis for clinical detection of tumors by positron emission tomography (PET). PET depends on the fact that tumors exhibit higher uptake of ¹⁸F-deoxyglucose. Since ¹⁸F-deoxyglucose uptake inversely correlates with glucose concentrations in cultured cells (Haberkorn U, Morr I, Oberdorfer F, Bellemann M E, Blatter J, et al. (1994) Fluorodeoxyglucose uptake in vitro: aspects of method and effects of treatment with gemcitabine. J Nucl Med 35: 1842-1850), PET detection of ¹⁸F-deoxyglucose uptake in tumor cells may reflect glucose deprivation in tumors in cancer patients. Since glucose deficiency selects cells with oncogenic mutations in vitro (Yun J, Rago C, Cheong I, Pagliarini R, Angenendt P, et al. (2009) Glucose deprivation contributes to the development of KRAS pathway mutations in tumor cells. Science 325: 1555-1559), glucose deprivation in cancer cells, as demonstrated by the present inventors, may offer a proliferative advantage of cancer cells in vivo.

It has been reported that glucose-induced uH2B regulates expression of metabolic genes in yeast (Dong L, Xu C W (2004) Carbohydrates induce mono-ubiquitination of H2B in yeast. J Biol Chem 279: 1577-1580). It has also been shown that uH2B is required for DNA repair in yeast and mammalian cells (Chernikova S B, Dorth J A, Razorenova O V, Game J C, Brown J M (2010) Deficiency in Brel impairs homologous recombination repair and cell cycle checkpoint response to radiation damage in mammalian cells. Radiat Res 174: 558-565; Moyal L, Lerenthal Y, Gana-Weisz M, Mass G, So S, et al. (2011) Requirement of ATM-Dependent Monoubiquitylation of Histone H2B for Timely Repair of DNA Double-Strand Breaks. Mol Cell 41: 529-542; Nakamura K, Kato A, Kobayashi J, Yanagihara H, Sakamoto S, et al. (2011) Regulation of Homologous Recombination by RNF20-Dependent H2B Ubiquitination. Mol Cell 41: 515-528). Since glucose-induced uH2B was impaired in virtually all cancer cells from breast, colon, lung and additional 23 anatomic sites that have been tested by the present inventors, coupling of glucose deprivation with loss of uH2B may play an important role in regulating metabolic reprogramming as well as DNA repair in tumorigenesis.

In certain embodiments of the invention, methods are provided for increasing mono-ubiquitination of a histone in a cell. In certain embodiments, the histone is histone H2B. In certain embodiments, the method comprises upregulating or increasing a ubiquitin ligase. In certain embodiments, a ubiquitin ligase that is positively modulated is at least one of RNF20, RNF40, WAC, BUR, PAF, HR6A/B(RAD6), UbcH6 and other positive regulators of uH2B.

In some embodiments, the cell is a cancer cell. In certain embodiments, the cancer is a carcinoma. Exemplary types of cancer cells include, but are not limited to, breast, prostate, colon, and lung tumor cells.

In some embodiments, the positive modulator of mono-ubiquitination of the histone comprises an inhibitor of a deubiquitinating enzyme or a deubiquitinating enzyme cofactor. The inhibitor may directly or indirectly inhibit a deubiquitinating enzyme or a deubiquitinating enzyme cofactor. In certain embodiments, the deubiquitinating enzyme is a ubiquitin-specific protease. In certain embodiments, the ubiquitin-specific protease is at least one of ubiquitin specific peptidase 22 (USP22), USP7/HAUSP, or another deubiquitinase that can remove the ubiquitin moiety of histone H2B.

In yeast, Ubp8 (yeast homolog of USP22) itself has very little deubiquitinase activity. The deubiquitinase activity is activated by three interacting cofactors (Sgf11, Sgf73 and Sus1) of the SAGA complex (Samara N L, Datta A B, Berndsen C E, Zhang X, Yao T, et al. (2010) Structural insights into the assembly and function of the SAGA deubiquitinating module, Science 328: 1025-1029; Kohler A, Zimmerman E, Schneider M, Hurt E, Zheng N (2010) Structural basis for assembly and activation of the heterotetrameric SAGA histone H2B deubiquitinase module. Cell 141: 606-617). The human cofactors of USP22 are ATXN7L3 (human homolog of Sgf1), ATXN7 (human homolog of Sgf73) and ENY2 (human homolog of Sus1), respectively. Therefore, in certain embodiments, to inhibit USP22 activity, one may use an inhibitor of USP22 per se. In other embodiments, inhibitors of the interaction of USP22 with one or more of cofactors ATXNL73, ATXN7 or ENY2 may be used to inhibit the function of USP22 In other embodiments, inhibitors of the cofactors, e.g. ATXNL73, ATXN7 or ENY2 are used. In certain embodiments, small molecules that block the interaction of USP22 with at least one of ATXNL73, ATXN7 or ENY2 may be used. In certain embodiments, small molecules that inhibit USP22, ATXNL73, ATXN7 or ENY2 may be used. In certain embodiments, more than one small molecule is used, wherein the small molecule is an inhibitor of USP22, ATXNL73, ATXN7 or ENY2. In certain embodiments, two small molecule inhibitors are used.

In certain embodiments of the invention, methods are provided for treatment of cancer comprising administration to a subject in need of treatment a composition comprising a negative modulator (inhibitor) of a deubiquitinating enzyme or a deubiquitinating enzyme cofactor. In certain embodiments, the deubiquitinating enzyme is a ubiquitin-specific protease that inhibits mono-ubiquitination of a histone. Preferably, the inhibitor of the deubiquitinating enzyme is administered in an amount effective to positively modulate mono-ubiquitination of a histone in cancer cells in the subject. In certain embodiments, the histone is histone H2B. In certain embodiments, the ubiquitin-specific protease that is inhibited by the negative modulator is at least one of USP22, USP7/HAUSP or another deubiquitinase that can remove the ubiquitin moiety of histone H2B. In certain embodiments, at least one inhibitor of the interaction of USP22 with one or more of ATXNL73, ATXN7 or ENY2 may be used. In other embodiments, inhibitors of the cofactors, e.g. ATXNL73, ATXN7 or ENY2 are used. In certain embodiments, an inhibitor of USP22 interaction with at least one of ATXNL73, ATXN7 or ENY2 may be a small molecule. In certain embodiments, small molecules that inhibit USP22, ATXNL73, ATXN7or ENY2 may be used. In certain embodiments, more than one small molecule is used, wherein the small molecule is an inhibitor of USP22, ATXNL73, ATXN7 or ENY2. In certain embodiments, two small molecule inhibitors are used.

In certain embodiments of the invention, methods are provided for restoring glucose levels in and around cells. While not intending to be bound by any theory of operation, reversal of glucose deficiency in tumor cells may lead to an increase of mono-ubiquitination of a histone in a cell. In certain embodiments, the histone is histone H2B. In certain embodiments, suitable agents include, but are not limited to, positive modulators of glucose metabolism and agents that increase glucose transport into cells.

In some embodiments, the cell is a cancer cell. In certain embodiments, the cancer is a carcinoma. Exemplary types of cancer cells include, but are not limited to, breast, prostate, colon, and lung tumor cells.

Another embodiment provides a composition comprising at least one negative modulator (inhibitor) of a deubiquitinating enzyme or a deubiquitinating enzyme cofactor. Such a composition may be used in connection with various methods according to aspects of the invention. In certain embodiments, the deubiquitinating enzyme is a ubiquitin-specific protease that inhibits mono-ubiquitination of a histone. In certain embodiments, the histone is histone H2B. In certain preferred embodiments, the composition comprises an inhibitor of least one of USP22, USP7/HAUSP or another deubiquitinase that can remove the ubiquitin moiety of histone H2B. In certain embodiments, the composition comprises at least one inhibitor of the interaction of USP22 with one or more of ATXNL73, ATXN7 or ENY2. In other embodiments, inhibitors of the cofactor, e.g., ATXNL73, ATXN7 or ENY2 are used. In certain embodiments, the at least one inhibitor of USP22 interaction with one or more of ATXNL73, ATXN7 or ENY2 may be a small molecule. In certain embodiments, small molecules that inhibit USP22, ATXNL73, ATXN7 or ENY2 may be used. In certain embodiments, more than one small molecule is used, wherein the small molecule is an inhibitor of USP22, ATXNL73, ATXN7 or ENY2. In certain embodiments, two small molecule inhibitors are used.

Another embodiment provides a method of treatment for cancer comprising administration to a subject in need of treatment a composition comprising a negative modulator (inhibitor) of glucose metabolism in an amount effective to kill cancer cells in the subject. While not intending to be bound by any theory of operation, the observation that cancer cells are already glucose-deprived, as demonstrated by the present inventors, indicates that a glycolytic inhibitor may therefore have more detrimental effect on cancer cells than on normal cells. Thus, an inhibitor of glucose metabolism may preferentially kill cancer cells while leaving normal cells intact. In certain embodiments, the cancer is carcinoma. In certain embodiments, the cancer is selected from the group consisting of breast, prostate, colon, and lung cancer.

In another embodiment, a composition is provided comprising a negative modulator of glucose metabolism. Inhibitors of glucose metabolism suitable for use in compositions and methods according to embodiments of the invention include, but are not limited to: Phloretin, 2-deoxyglucose, 3-bromopyruvate, lonidamine, 3PO, CAP-232/TLN-232, Dichloroacetate, FX11, Oxamate, Amino oxyacetate, AZD-3965, 5-Dehydroepiandrosterone [DHEA], Oxythiamine, Tarvagenix, 6-Diazo-5-oxo-L-norleucine, 968 (Cornell University), BPTES, GSK837149A (GSK), C75, CPI-613 (Cornerstone Pharmaceutical), Metformin, MPC-9528 (Myrexis), disulfiram, ethylene glycol poisoning, fluoride, iodoacetate, mercury and arsenite (As₃O), sulfhydryl, and pentavalent arsenic AsO₄.

The therapeutic agents of the present invention may be used alone or in combination with other cancer therapies including, for example, chemotherapy, radiation therapy, immunotherapy and gene therapy.

In another embodiment, the method of treatment further comprises the administration of a second therapeutic agent. In a preferred embodiment, the second therapeutic agent is an anticancer agent. In certain embodiments, the second agent may be administered before, after, or concurrently with the negative modulator of a deubiquitinating enzyme or a modulator of glucose metabolism.

Negative modulators of a deubiquitinating enzyme, e.g. UPS22, and negative modulators of deubiquitinating enzyme cofactors, e.g. ATXNL73, ATXN7 and ENY2, include, but are not limited to: antibodies, aptamers, antisense oligonucleotides, interfering RNA, and small molecule inhibitors. These agents are suitable for various methods described herein.

Exemplary inhibitors of deubiquitinating enzyme, e.g. UPS22, and exemplary inhibitors of deubiquitinating enzyme cofactors, e.g. ATXNL73, ATXN7 and ENY2, include, but are not limited to:

a) antibodies that immunoreact (bind) with a deubiquitinating enzyme or deubiquitinating enzyme cofactor (also known as deubiquitinating enzyme antibodies or anti-deubiquitinating enzyme antibodies);

(b) fragments of (a) that retain antigen binding activity;

(c) polypeptides that comprise an antigen binding domain of (a) or (b) and that bind the antigen;

(d) antisense oligonucleotides that inhibit deubiquitinating enzyme or deubiquitinating enzyme cofactor transcription or translation;

(e) aptamers that inhibit a deubiquitinating enzyme or deubiquitinating enzyme cofactor;

(f) short interfering RNAs (siRNA, RNAi) that inhibit translation of deubiquitinating enzyme or a cofactor.

(g) small molecule inhibitors of a deubiquitinating enzyme or deubiquitinating enzyme cofactor; and

(f) combinations thereof.

Antibodies

Anti-deubiquitinating enzyme or deubiquitinating enzyme cofactor antibodies, including, for example, monoclonal, polyclonal, human, humanized and bispecific antibodies may be used in the methods described herein. Polyclonal or monoclonal therapeutic anti-deubiquitinating enzyme antibodies useful in practicing this invention may be prepared in laboratory animals or by recombinant DNA techniques using the methods known in the art, or may be obtained commercially Polyclonal antibodies to a deubiquitinating enzyme or cofactor molecule or a fragment thereof containing the target amino acid sequence generally are raised in animals by multiple subcutaneous (sc) or intraperitoneal (ip) injections of the deubiquitinating enzyme molecule in combination with an adjuvant such as Freund's adjuvant (complete or incomplete). To enhance immunogenicity, it may be useful to first conjugate the deubiquitinating enzyme or cofactor molecule or a fragment containing the target amino acid sequence of to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOC₁, or R¹N=C=NR, where R and R¹ are different alkyl groups. Alternatively, deubiquitinating enzyme- or cofactor-immunogenic conjugates can be produced recombinantly as fusion proteins.

Animals may be immunized against the immunogenic deubiquitinating enzyme or cofactor conjugates or derivatives (such as a fragment containing the target amino acid sequence) by combining about 1 mg or about 1 microgram of conjugate (for rabbits or mice, respectively) with about 3 volumes of Freund's complete adjuvant and injecting the solution intradermally at multiple sites. Approximately 7 to 14 days later, animals are bled and the serum is assayed for anti-deubiquitinating enzyme or cofactor titer. Animals are boosted with antigen repeatedly until the titer plateaus. Preferably, the animal is boosted with the same deubiquitinating enzyme or cofactor molecule or fragment thereof as was used for the initial immunization, but conjugated to a different protein and/or through a different cross-linking agent. In addition, aggregating agents such as alum may be used in the injections to enhance the immune response.

Monoclonal antibodies may be prepared by recovering spleen cells from immunized animals and immortalizing the cells in conventional fashion, e.g. by fusion with myeloma cells. The clones are then screened for those expressing the desired antibody.

Preparation of antibodies using recombinant DNA methods, such as the phagemid display method, may be accomplished by methods known in the art and may be performed using commercially available kits, as for example, the Recombinant Phagemid Antibody System available from Pharmacia (Uppsala, Sweden), or the SurfZAP™. phage display system (Stratagene Inc., La Jolla, Calif.). Human antibodies may also be prepared with yeast display methods as disclosed, for example, by E. T. Boder and K. D. Wittrup, Nat Biotech (1997) 553-557.

Preferably, antibodies for administration to humans may be “humanized”, or chimeric, i.e. made to be compatible with the human immune system such that a human patient will not develop an immune response to the antibody. Even more preferably, human antibodies prepared using methods such as those described for example, in Lonberg, et al., Nature Genetics, 7:13-21 (1994) are preferred for therapeutic administration to patients. Further methods for making antibodies are disclosed in U.S. Patent Application Publication 20110076761.

The term “antigen binding domain” or “antigen binding region” refers to that portion of the selective binding agent (such as an antibody molecule) which contains the specific binding agent amino acid residues that interact with an antigen and confer on the binding agent its specificity and affinity for the antigen. In an antibody, the antigen binding domain is commonly referred to as the “complementarity determining region”, or “CDR.”

Aptamers

Recent advances in the field of combinatorial sciences have identified short polymer sequences with high affinity and specificity to a given target. For example, SELEX technology has been used to identify DNA and RNA aptamers with binding properties that rival mammalian antibodies. The field of immunology has generated and isolated antibodies or antibody fragments which bind to a myriad of compounds and phage display has been utilized to discover new peptide sequences with very favorable binding properties. Based on the success of these molecular evolution techniques, ligands can be created which bind to a deubiquitinating enzyme or cofactor. In each case, a loop structure is often involved with providing the desired binding attributes as in the case of: aptamers which often utilize hairpin loops created from short regions without complimentary base pairing, naturally derived antibodies that utilize combinatorial arrangement of looped hyper-variable regions and new phage display libraries utilizing cyclic peptides that have shown improved results when compare to linear peptide phage display results. Thus, high affinity ligands can be created and identified by combinatorial molecular evolution techniques. For the present invention, molecular evolution techniques can be used to isolate ligands specific for deubiquitinating enzymes or cofactors. For more on aptamers, see generally, Gold, L., Singer, B., He, Y. Y., Brody. E., “Aptamers As Therapeutic And Diagnostic Agents,” J. Biotechnol. 74:5-13 (2000).

Anti-Sense Molecules

Another class of deubiquitinating enzymeor cofactor inhibitors useful in certain embodiments is isolated antisense nucleic acid molecules that can hybridize to, or are complementary to, the nucleic acid molecule comprising a deubiquitinating enzyme or cofactornucleotide sequence, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). (See, for example, Uhlmann, et al. Antisense oligonucleotides: A new therapeutic principle. Chemical Reviews 1990, 90: 543-584; Crooke, et al. “Antisense Research and Applications”, CRC Press (1993); Mesmaekar, et al. “Antisense oligonucleotides,”, Acc. Chem. Res. 1995, 28: 366-374; Stein. “The experimental use of antisense oligonucleotides: a guide for the perplexed.” J. Clin. Invest. 2001, 108, 641-644, and U.S. Pat. Nos. 6,117,992; 6,127,121; 6,235,887; 6,232,463; 6,579,704; 5,596,091; 6,031,086 and 6,117,992, the disclosures of which are incorporated herein by reference in their entireties). In certain embodiments, antisense nucleic acid molecules may comprise a sequence complementary to at least about 10, 25, 50, 100, 250 or 500 nucleotides or an entire deubiquitinating enzyme or cofactor coding strand, or to only a portion thereof. Nucleic acid molecules encoding fragments, homologs, derivatives and analogs of deubiquitinating enzyme or cofactor antisense nucleic acids complementary to a deubiquitinating enzyme nucleic acid sequence may be used.

In one embodiment, an antisense nucleic acid molecule is antisense to a “coding region” of the coding strand of a nucleotide sequence encoding a deubiquitinating enzyme or cofactor protein. The term “coding region” refers to the region of the nucleotide sequence comprising codons that are translated into amino acid residues. In another embodiment, the antisense nucleic acid molecule is antisense to a “conceding region” of the coding strand of a nucleotide sequence encoding the target protein. The term “conceding region” refers to 5′ and 3′ sequences that flank the coding region and that are not translated into amino acids (i.e., also referred to as 5′ and 3′ untranslated regions).

The antisense nucleic acid molecule can be complementary to the entire coding region of a deubiquitinating enzyme or cofactor mRNA, but more preferably is an oligonucleotide that is antisense to only a portion of the coding or noncoding region of a deubiquitinating enzyme mRNA. For example, the antisense oligonucleotide can be complementary to the region surrounding the translation start site of a deubiquitinating enzyme or cofactor mRNA. An antisense oligonucleotide can be, for example, about 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include, without limitation: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridin-e, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiour-acil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

In certain embodiments, antisense nucleic acid molecules may be administered to a subject or generated in situ such that they hybridize with or bind to cellular mRNA and/or genomic DNA encoding a deubiquitinating enzyme or cofactor to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens).

In yet another embodiment, the antisense nucleic acid molecule of the invention is an alpha-anomeric nucleic acid molecule. An alpha-anomeric nucleic acid molecule forms specific double-stranded hybrids with complementary RNA in which, contrary to the usual alpha-units, the strands run parallel to each other. See, e.g., Gaultier, et al., Nucl. Acids Res., 15:6625-6641 (1987). The antisense nucleic acid molecule can also comprise a 2′-o-methylribonucleotide (see, e.g., Inoue, et al. Nucl. Acids Res., 15:6131-6148 (1987)) or a chimeric RNA-DNA analogue (see, e.g., Inoue, et al., FEBS Lett., 215:327-330 (1987)).

Production and delivery of antisense molecules may be facilitated by providing a vector comprising an anti-sense nucleotide sequence complementary to at least a part of the deubiquitinating enzyme DNA sequence. According to a yet further aspect of the invention such a vector comprising an anti-sense sequence may be used to inhibit deubiquitinating enzyme expression.

RNA Interference

In certain embodiments, use of RNA interference to inactivate or modulate expression of a deubiquitinating enzyme or cofactor is provided. RNA interference is described in U.S. Patent Application Publication No. 2002-0162126, and Hannon, G., J. Nature, 11:418:244-51 (2002).

RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene.

Mammalian cells have at least two pathways that are affected by double-stranded RNA (dsRNA). In the RNAi (sequence-specific) pathway, the initiating dsRNA is first broken into short interfering (si) RNAs. The siRNAs have sense and antisense strands of about 21 nucleotides that form approximately 19 nucleotide siRNAs with overhangs of two nucleotides at each 3′ end. Short interfering RNAs provide the sequence information that allows a specific messenger RNA to be targeted for degradation. In contrast, the nonspecific pathway is triggered by dsRNA of any sequence, as long as it is at least about 30 base pairs in length. Longer dsRNAs appear to be required to induce the nonspecific pathway and, accordingly, dsRNAs shorter than about 30 bases pairs are preferred to effect gene repression by RNAi (see Hunter et al. (1975) J. Biol. Chem. 250: 409-17; Manche et al. (1992) Mol. Cell Biol. 12: 5239-48; Minks et al. (1979) J. Biol. Chem. 254: 10180-3; and Elbashir et al. (2001) Nature 411: 494-8).

The double stranded oligonucleotides used to affect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides may include 3′ overhang ends. Exemplary 2-nucleotide 3′ overhangs may be composed of ribonucleotide residues of any type and may even be composed of 2′-deoxythymidine resides. Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable to the skilled artisan. The dsRNAs may be synthesized chemically or produced in vitro or in vivo using appropriate expression vectors, or may be obtained commercially Exemplary synthetic RNAs include 21 nucleotide RNAs chemically synthesized using methods known in the art. Synthetic oligonucleotides are preferably deprotected and gel-purified using methods known in the art (see e.g. Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species may be designed to include a portion of nucleic acid sequence represented in a deubiquitinating enzyme nucleic acid.

The dsRNA need only be sufficiently similar to the target mRNA such that it mediates RNAi. The dsRNA may have at least 50%, preferably at least 70%, more preferably at least 80%, and most preferably at least 90% sequence identity with the target.

A preferred interfering RNA of the present invention is a siRNA, particularly small hairpin RNA (shRNA). siRNA, particularly shRNA, mediate the post-transcriptional process of gene silencing by double stranded RNA (dsRNA) that is homologous in sequence to the silenced RNA. siRNA according to the present invention preferably comprises a sense strand of 15-30 nucleotides, and an antisense strand of 15-30 nucleotides complementary to the sense strand. The siRNA preferably further comprises a loop region linking the sense and the antisense strand.

The siRNAs may be modified by methods known in the art for example by modified internucleoside linkages, modified nucleic acid bases, modified sugars and/or chemical linkage the siRNA to one or more moieties or conjugates.

The specific sequence utilized in design of the oligonucleotides may be any contiguous sequence of nucleotides contained within the expressed gene message of deubiquitinating enzyme or cofactor. Programs and algorithms known in the art may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588.

The RNA oligonucleotides may be introduced into a cell by methods known in the art for introducing ribonucleic acids into animal cells and disclosed for example in U.S. Patent Application Publication Nos. 20100120891 and 20110065908. For example, transfection with using carrier compositions such as liposomes, are known in the art—e.g. Lipofectamine 2000 (Life Technologies). Transfection of dsRNA oligonucleotides for targeting endogenous genes may be carried out using Oligofectamine (Life Technologies). Nanoparticles such as the cationic polymer polyethyleneimine (PEI) may also be used to deliver siRNA to target cells. The RNA oligonucleotides may also be delivered by viral transduction utilizing, for example, an adenoviral, lentiviral, baculoviral, or adeno-associated viral vector. Other nonlimiting examples include the use of modified viral particles and implantable drug-releasing biodegradable microspheres.

Further compositions, methods and applications of RNAi technology are provided in U.S. Pat. Nos. 6,278,039, 5,723,750 and 5,244,805.

Formulations

Formulations of the compositions useful in certain embodiments such as polypeptides, polynucleotides, or antibodies may be prepared for storage by mixing the selected composition having the desired degree of purity with optional physiologically pharmaceutically-acceptable carriers, excipients, or stabilizers (Remington's Pharmaceutical Sciences, 18th edition, A. R. Gennaro, ed., Mack Publishing Company (1990)) in the form of a lyophilized cake or an aqueous solution. Acceptable carriers, excipients or stabilizers are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

Compositions to be used for in vivo administration should be sterile. This is readily accomplished by filtration through sterile filtration membranes, prior to or following lyophilization and reconstitution. The composition for parenteral administration ordinarily will be stored in lyophilized form or in solution.

Therapeutic compositions generally are placed into a container having a sterile access port, for example, an intravenous solution bag or vial having a stopper pierceable by a hypodermic injection needle. The route of administration of the composition is in accord with known methods, e.g. oral, injection or infusion by intravenous, intraperitoneal, intracerebral, intramuscular, intraocular, intraarterial, or intralesional routes, or by sustained release systems or implantation device. Where desired, the compositions may be administered continuously by infusion, bolus injection or by implantation device.

An effective amount of the compositions to be employed therapeutically will depend, for example, upon the therapeutic objectives, the route of administration, and the condition of the patient. Accordingly, it may be necessary for the therapist to titer the dosage and modify the route of administration as required to obtain the optimal therapeutic effect. A typical daily dosage may range from about 1 μg/kg to up to 100 mg/kg or more, depending on the factors mentioned above. Typically, a clinician will administer the composition until a dosage is reached that achieves the desired effect. The progress of this therapy is easily monitored by conventional assays designed to evaluate blood glucose levels or other particular conditions of interest in a particular subject.

Pharmaceutical compositions may be produced by admixing a pharmaceutically effective amount of protein with one or more suitable carriers or adjuvants such as water, mineral oil, polyethylene glycol, starch, talcum, lactose, thickeners, stabilizers, suspending agents, etc. Such compositions may be in the form of solutions, suspensions, tablets, capsules, creams, salves, ointments, or other conventional forms.

In certain embodiments, compounds are formulated with pharmaceutically acceptable diluents, adjuvants, excipients, or carriers. The phrase “pharmaceutically or pharmacologically acceptable” refers to molecular entities and compositions that do not produce adverse, allergic, or other untoward reactions when administered to an animal or a human, e.g., orally, topically, transdermally, parenterally, by inhalation spray, vaginally, rectally, or by intracranial injection. The term parenteral as used herein includes subcutaneous injections, intravenous, intramuscular, intracistemal injection, or infusion techniques. Administration by intravenous, intradermal, intramusclar, intramammary, intraperitoneal, intrathecal, retrobulbar, intrapulmonary injection and/or surgical implantation at a particular site is contemplated as well.) Generally, this will also entail preparing compositions that are essentially free of pyrogens, as well as other impurities that could be harmful to humans or animals. The term “pharmaceutically acceptable carrier” includes any and all solvents, dispersion media, coatings, antibacterial and antifungal agents, isotonic and absorption delaying agents, liposomes, capsids, nanocapsules, microcapsules and the like. The use of such media and agents for pharmaceutically active substances is well known in the art.

The pharmaceutical forms suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Routes of Administration

In certain embodiments, the present invention provides a method of treating a subject comprising administration of a composition. As used herein, the term “subject” is used to mean an animal, preferably a mammal, including a human. The terms “patient” and “subject” may be used interchangeably.

The therapeutic compositions may be administered by any route that delivers an effective dosage to the desired site of action, with acceptable (preferably minimal) side-effects. Numerous routes of administration of agents are known, for example, oral, rectal, transmucosal, or intestinal administration; parenteral delivery, including intramuscular, subcutaneous, intramedullary injections, as well as intrathecal, intraperitoneal, intranasal, cutaneous or intradermal injections; inhalation, and topical application.

Therapeutic dosing is achieved by monitoring therapeutic benefit and monitoring to avoid side-effects. Preferred dosage provides a maximum localized therapeutic benefit with minimum local or systemic side-effects. Suitable human dosage ranges for the polynucleotides or polypeptides can be extrapolated from these dosages or from similar studies in appropriate animal models. Dosages can then be adjusted as necessary by the clinician to provide maximal therapeutic benefit for human subjects.

When a therapeutically effective amount of a composition of the present invention is administered by e.g., intradermal, cutaneous or subcutaneous injection, the composition is preferably in the form of a pyrogen-free, parenterally acceptable aqueous solution. The preparation of such parenterally acceptable protein or polynucleotide solutions, having due regard to pH, isotonicity, stability, and the like, is within the skill in the art. A preferred composition should contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The composition of the present invention may also contain stabilizers, preservatives, buffers, antioxidants, or other additives known to those of skill in the art. The agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

The compositions of the invention may be in the form of a complex of the protein(s) or other active ingredient of present invention along with protein or peptide antigens.

The composition may further contain other agents which either enhance the activity of the protein or other active ingredient or complement its activity or use in treatment. Such additional factors and/or agents may be included in the pharmaceutical composition to produce a synergistic effect with protein or other active ingredient, or to minimize side effects.

Techniques for formulation and administration of the therapeutic compositions of the instant application may be found in “Remington's Pharmaceutical Sciences,” Mack Publishing Co., Easton, Pa., latest edition. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

Additional embodiments of the invention provide methods of assaying the glucose content of a cell or tissue comprising detecting the level of uH2B in the cell or tissue. In certain embodiments, the level of uH2B is detected using antibodies that immunoreact (bind) with a uH2B (also known as uH2B antibodies or anti- uH2B antibodies). uH2B antibodies may be prepared by suitable known methods of antibody technology, including those described herein above.

The following examples serve to further illustrate the present invention.

### Example 1

### Materials and Methods

Cell Lines, Culture Media, Chemicals and Antibodies

U87 MG human glioblastoma (grade IV) cells and MCF7 (ATCC) were maintained in high-glucose Dulbecco's modified Eagle's medium (DMEM) (4.5 g/L glucose, 0.584 g/L glutamine and 110 mg/L pyruvate, (catalog # 11995, Gibco) supplemented with 10% fetal bovine serum (catalog # A15-351, PAA Laboratories) and 1% penicillin/streptomycin (P/S) at 37° C. in a humidified atmosphere of 95% air and 5% CO₂. LnCap and HCT116 (ATCC) were cultured in RPMI 1640 medium (catalog # 11875, Gibco) supplemented with 10% FBS and 1% P/S at 37° C. in a humidified atmosphere of 95% air and 5% CO₂. FBS (10 ml) was dialyzed against PBS (pH 7.4, 2×1 liter) at 4° C. for 48 hrs. The dialyzed FBS (dFBS) was filtered thorough 0.22 μm filter unit (Millipore) and stored at 4° C. until use.

Glucose minus DMEM (catalog # 11966, Gibco) contained 584 mg/L L-glutamine but no glucose. Glucose minus RPMI 1640 (catalog # 22400, Gibco) contained L-glutamine but no glucose. Mouse monoclonal antibody specific to ubiquitinated histone H2B at K120 was obtained from Medimabs (catalog # MM-0029-P) (Minsky N, et al. (2008) Monoubiquitinated H2B is associated with the transcribed region of highly expressed genes in human cells, Nat Cell Biol 10: 483-488). Histone H2B antibody (ChIP grade) was obtained from Abcam (catalog # ab1790). USP22 antibody was obtained from Sigma (catalog # HPA044980). Beta-actin antibody was obtained from Abcam (catalog # ab8224). Peroxidase-conjugated immuno pure goat anti-mouse IgG (H+L) (catalog # 31430) and peroxidase-conjugated immuno pure goat anti-rabbit IgG (H+L) (catalog # 31460) were obtained from Pierce.

Glucose Analysis of Human Normal Tissue and Tumor Samples

Matched human tumor and normal tissue specimens of the same tissue origins (the samples were paired, unfixed and frozen) were obtained from BioChain (Breast cancer, catalog # P8235090-PP; and colon cancer, catalog # P8235090-PP). Additional paired tumor and normal specimens (frozen/unfixed breast, prostate and colon tumor/normal tissues CP5656504; CP565671; CP5655651; CP565424; CP5655718; CP565608) were obtained from Origene. Glucose was assayed described by Dong and Xu ((2004) Carbohydrates induce mono-ubiquitination of H2B in yeast. J Biol Chem 279: 1577-1580). Protein concentration was estimated with CB-X Protein Assay Kit (catalog # 786-12X, G Biosciences).

Analysis of Glucose-Regulated uH2B in Cultured Tumor Cells and Yeast

U87 (Glioblastoma), MCF7 (breast cancer), LnCap (prostate cancer), and HCT116 (colon cancer) were cultured in 60 mm dishes with high-glucose DMEM or RPMI 1640 supplemented with 10% FBS and 1% P/S until they were 40-60% confluent. After the media were removed, cells were rinsed twice with phosphate buffered saline (PBS) and subsequently incubated with glucose minus medium DMEM (catalog # 11966) or RPMI 1640 (catalog # 22400) supplemented with 10% dialyzed FBS and 1% P/S and 0%, 0.045% or 0.450% glucose for 24, 40, or 48 hrs. Although some cells became detached during the glucose-minus medium incubation, virtually all of the detached cells excluded Trypan Blue (data not shown), indicating that they were alive.

To collect both attached and detached cells, the attached cells were scraped in glucose minus medium with cell scrapers (catalog # 353085, BD) and the cell suspension collected by centrifugation at 200 g for 2 min. The cell pellets were boiled in 4X SDS-PAGE sample buffer at 100° C. for 5 min. Protein concentration was estimated with CB-X Protein Assay Kit (catalog #786-12X, G Biosciences) and normalized by Western blotting. For yeast analysis, stationary phase (SP) yeast (Y117), which contained FLAG-tagged H2B as the sole source of H2B, was incubated with different amounts of glucose for 1 hr and harvested for Western blotting analysis (Dong L, and Xu CW ((2004) Carbohydrates induce mono-ubiquitination of H2B in yeast. J Biol Chem 279: 1577-1580). The intensity of Western blotting signals was estimated with Image J.

To correlate Western analysis with immunohistochemical analysis, half of the harvested cells were formalin-fixed and paraffin-embedded. Specifically, cell suspensions were centrifuged at 200 g for 2 min and washed once with PBS. Washed cells were collected by centrifugation and re-suspended by 1:10 buffer diluted formalin at room temperature for 24 hrs, subsequently paraffin-embedded and immunohistochemically stained.

Immunohistochemical Analysis of Clinical Tumor Specimens

Breast, colon and lung tumor arrays were obtained from Pantomics (catalog # BRC962, COC962 and LUC962). Tumor arrays from 27 anatomic sites were obtained from BioChain (catalog # Z7020082, lot # B412135). Mach 4 Universal HRP-Polymer Detection Kit (Biocare Medical, LLC) were used for immunohistochemical analyses with 200×dilution of the antibody raised against a synthetic branch peptide of ubiquitinated histone H2B at K120 (Minsky N, et al. (2008) Monoubiquitinated H2B is associated with the transcribed region of highly expressed genes in human cells. Nat Cell Biol 10: 483-488) (Medimabs, catalog # MM-0029-P) or histone H2B antibody (Abcam, catalog # ab1790). All images (40×) were captured and analyzed with an Aperio scanner (USC).

### Example 2

### Glucose Levels In Tumors Are Lower Than Those of Normal Tissues of the Same Tissue Sites

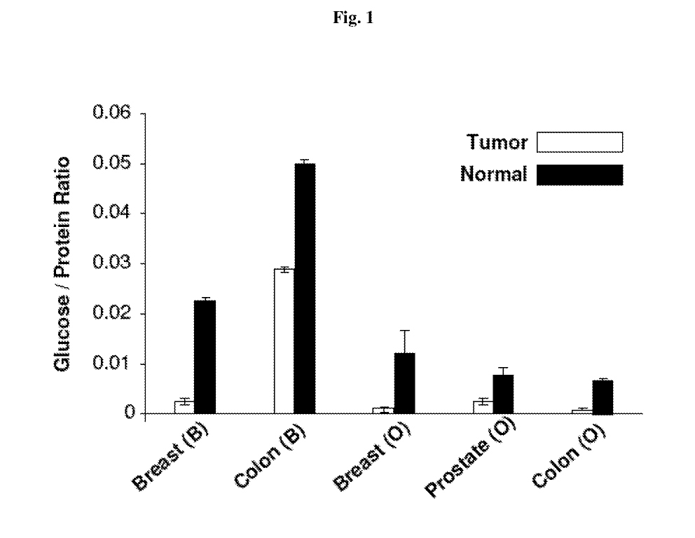

To determine glucose levels in human tumors and normal tissues, the glucose contents of matched clinical tumor specimens and normal tissues of the same tissue origins were assayed. Because of the inherent variability of clinical tissue specimens (Jackson D H, Banks R E (2010) Banking of clinical samples for proteomic biomarker studies: a consideration of logistical issues with a focus on pre-analytical variation. Proteomics Clin Appl 4: 250-270), the glucose levels were normalized with the total protein from the matched specimens (Waltregny D, North B, Van Mellaert F, de Leval J, Verdin E, et al. (2004) Screening of histone deacetylases (HDAC) expression in human prostate cancer reveals distinct class I HDAC profiles between epithelial and stromal cells. Eur J Histochem 48: 273-290). Although it is difficult to estimate the total protein levels in cancer and normal cells as a result of the tumor heterogeneity, the total protein level in tumor interstitial fluid is comparable to that of normal subcutaneous fluid in xenograft models (Gullino P M, Clark S H, Grantham F H (1964) The Interstitial Fluid of Solid Tumors. Cancer Res 24: 780-794), indicating that the total protein levels are operationally useful for normalizing the glucose amounts in inherently-variant clinical tumor and matched normal tissue specimens. As shown in FIG. 1, the relative amounts of glucose from frozen and unfixed human breast, prostate and colon tumor specimens were much lower than those of normal cells of the same tissue sites, indicating that glucose may be deprived in the bulk tumor specimens. These results are consistent with the observation that lower amounts of glucose are detected in tumor veins than in tumor arteries in rats (Warburg O, Wind F, Negelein E (1927) The Metabolism of Tumors in the Body. J Gen Physiol 8: 519-530). These results are also in agreement with the observation that lower amounts of glucose are found in tumor interstitial fluid than in normal subcutaneous interstitial fluid in xenograft models (Gullino P M, Clark S H, Grantham F H (1964) The Interstitial Fluid of Solid Tumors. Cancer Res 24: 780-794). Moreover, these results are consistent with the finding that glucose levels, detected by low-resolution bioluminescence assays, are drastically increased in bulk tumor specimens that have been treated with chemotherapy or radiation in comparison to untreated tumors in a xenograft model for lung cancer (Broggini-Tenzer A, Vuong V, Pruschy M (2011) Metabolism of tumors under treatment: mapping of metabolites with quantitative bioluminescence. Radiother Oncol 99: 398-403).

### Example 3

### uH2B Is A Semi-Quantitative Histone Marker For Glucose

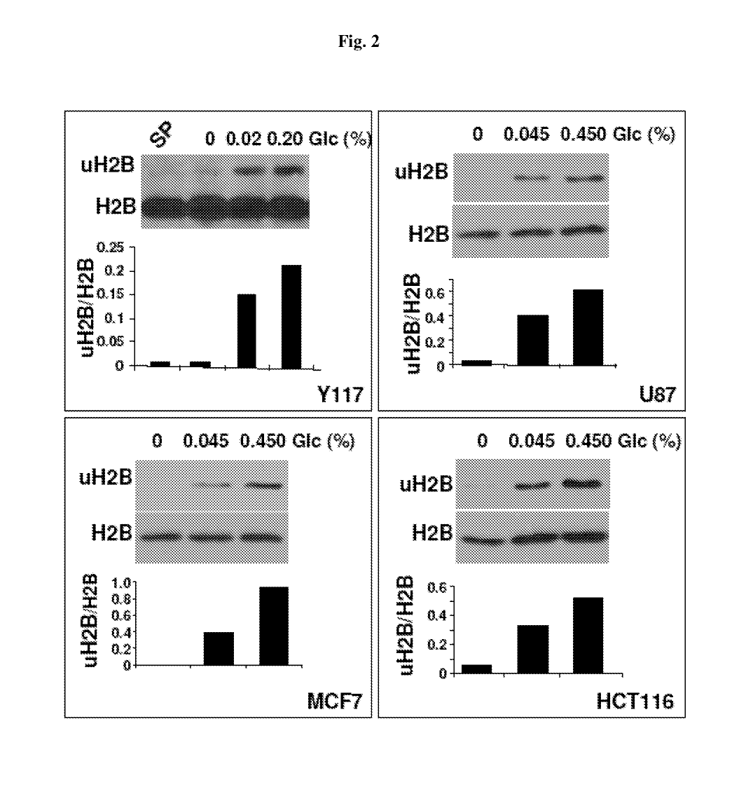

Tumors are typically heterogeneous organs with a microenvironment of various non-malignant cell types both within the tumor area and in their stromal environment (Weinberg R A (2007) The biology of Cancer. Garland Science). Therefore, a glucose marker should identify the cellular source of glucose deprivation in the bulk tumor specimens. It has been demonstrated that glucose is the sole nutrient inducer of mono-ubiquitination of histone H2B (uH2B) at K123 in yeast, and at its orthologous site K120 in human cells (Dong L, Xu C W (2004) Carbohydrates induce mono-ubiquitination of H2B in yeast. J Biol Chem 279: 1577-1580; Gao Z, Xu M S, Barnett T L, Xu C W (2011) Resveratrol induces cellular senescence with attenuated mono-ubiquitination of histone H2B in glioma cells. Biochem Biophys Res Commun 407: 271-276; Gao Z, Xu C W (2011) Glucose metabolism induces mono-ubiquitination of histone H2B in mammalian cells. Biochem Biophys Res Commun 404: 428-433), indicating that uH2B is an evolutionarily conserved chromatin marker for glucose. To test whether uH2B could be used as a semi-quantitative marker for glucose, U87 (glioblastoma), MCF7 (breast cancer) and HCT116 (colon cancer) were grown in various amounts of glucose spanning serum normal glucose levels. The levels of uH2B in these cells were then analyzed with an antibody specific to ubiquitinated histone H2B at K120 (Minsky N, Shema E, Field Y, Schuster M, Segal E, et al. (2008) Monoubiquitinated H2B is associated with the transcribed region of highly expressed genes in human cells. Nat Cell Biol 10: 483-488). As shown in FIG. 2, exposure to increasing levels of glucose resulted in a corresponding increase in levels of uH2B in tumor cells. In contrast, H2B levels remained unchanged in all samples. These data indicate that uH2B can be used as a semi-quantitative histone marker for glucose in tumor cells.

It has been reported that uH2B is not detectable in stationary phase yeast by Western blotting analysis (Dong L, Xu C W (2004) Carbohydrates induce mono-ubiquitination of H2B in yeast. J Biol Chem 279: 1577-1580). Using a yeast strain (Y117) with FLAG-H2B as the sole source of H2B, stationary phase Y117 were incubated in various amounts of glucose for 1 hr. As shown in FIG. 2, the uH2B levels also correlated with those of glucose, whereas H2B remained unchanged in all samples. Taken together, these data further indicate that uH2B is an evolutionarily conserved semi-quantitative marker for glucose in yeast and tumor cells.

uH2B levels as a function of glucose concentration were not linear in the range of glucose concentrations that were tested. While not seeking to be bound by any theory of operation, some of the glucose concentrations may have been at or above the saturation point for some of the cells; also, the observation may be due to the nonlinearity of Western blotting analysis, which was based on chemiluminescence/X-ray film imaging. Nevertheless, the results indicate that uH2B levels correlate semi-quantitatively with those of glucose in yeast and tumor cells.

### Example 4

### Immunohistochemical Staining of uH2B and H2B in Cultured Tumor Cells

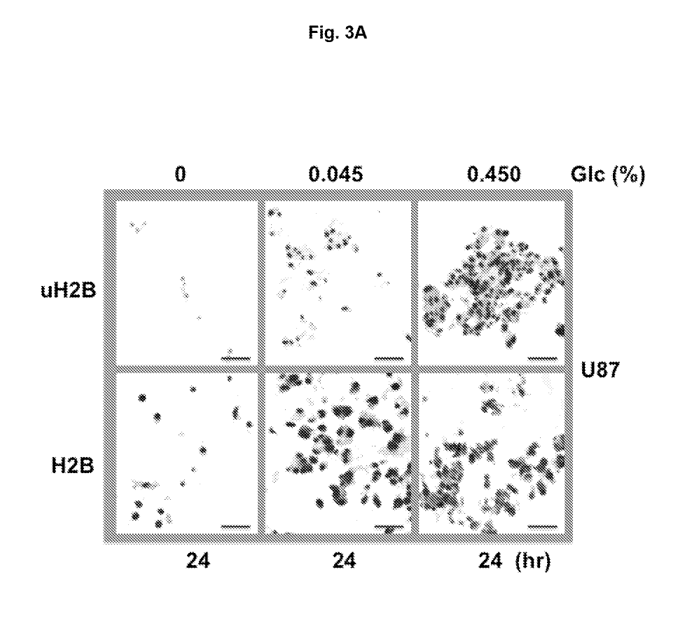

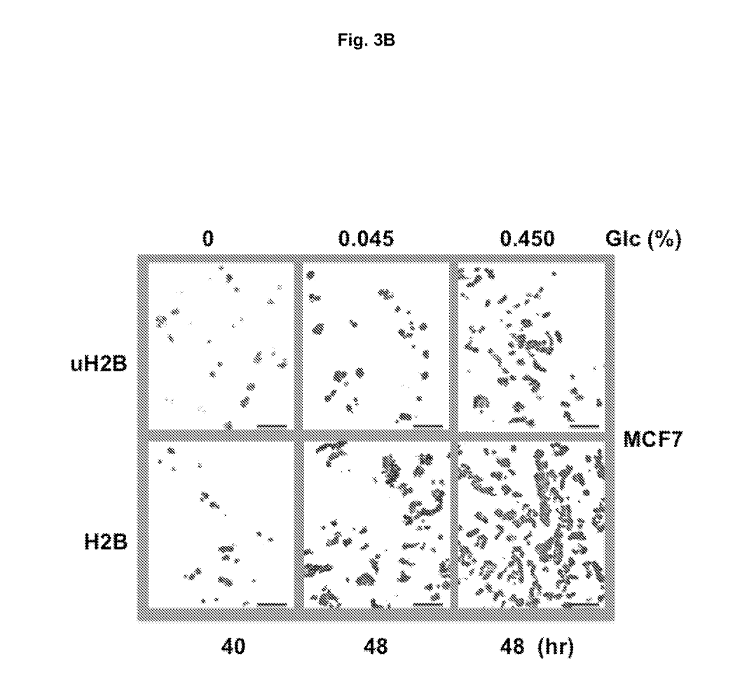

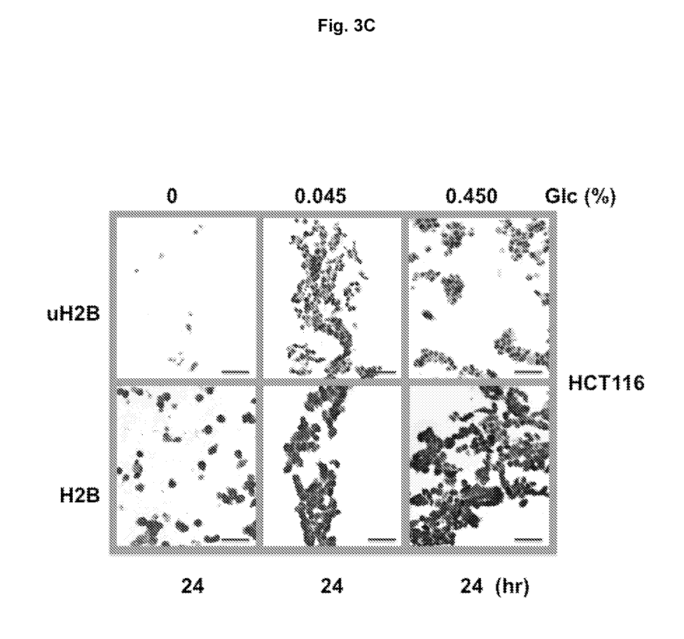

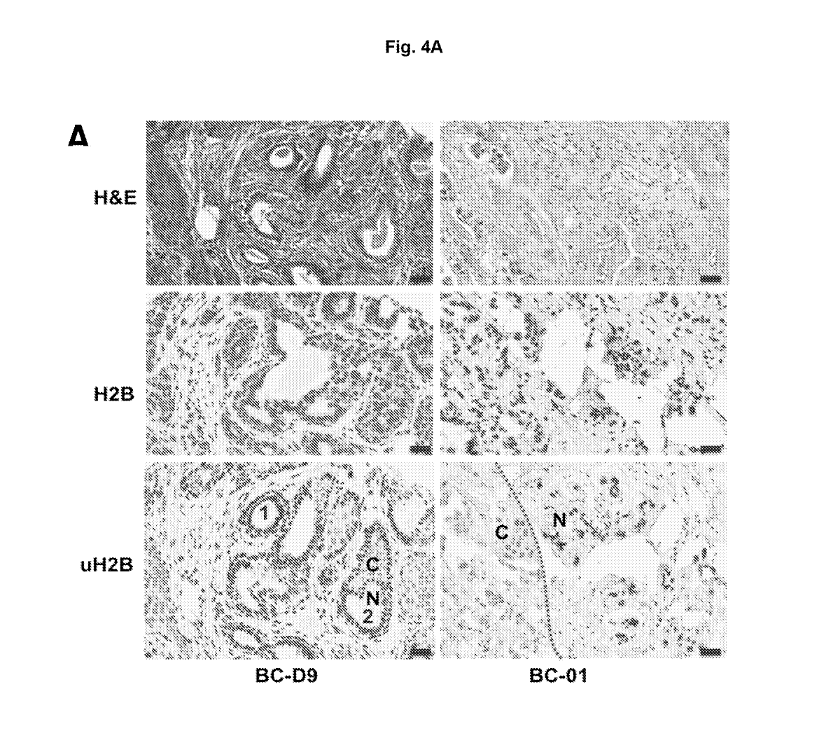

To determine whether the uH2B levels as a function of relative amounts of glucose could be detected by immunohistochemistry, the same batches of glucose-treated tumor cells used for the Western blotting analyses in FIG. 2 were formalin-fixed and paraffin-embedded. After hybridizing the cellblock sections with antibodies specific to either uH2B or H2B, and horseradish peroxidase-conjugated secondary antibody, the cells were counterstained with Hematoxylin. At least 1000 cells were examined for each sample. As shown in FIG. 3, uH2B levels detected by immunohistochemistry correlated with the amounts of glucose that the cells were exposed to. In contrast, H2B levels remained unchanged in all samples. Taken together, these data further demonstrate that glucose-induced uH2B may be used as a semi-quantitative chromatin marker for examining relative amounts of glucose in tumor specimens from cancer patients.

### Example 5

Glucose-Induced uH2B is Significantly Impaired in Cancer Cells of Patient Tumor Specimens Compared to Their Adjacent Stromal Tissues

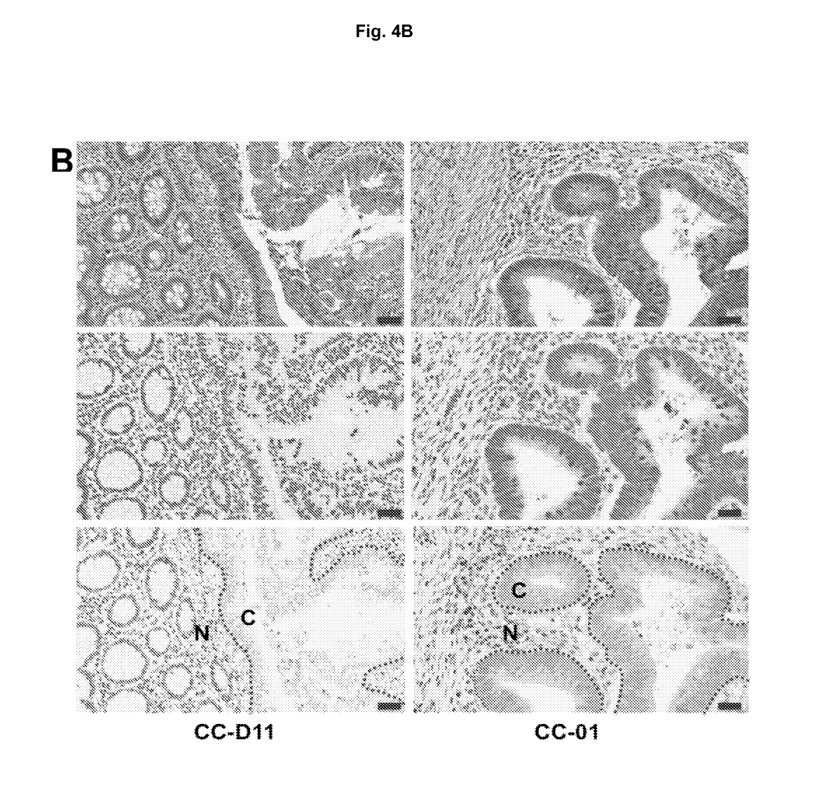

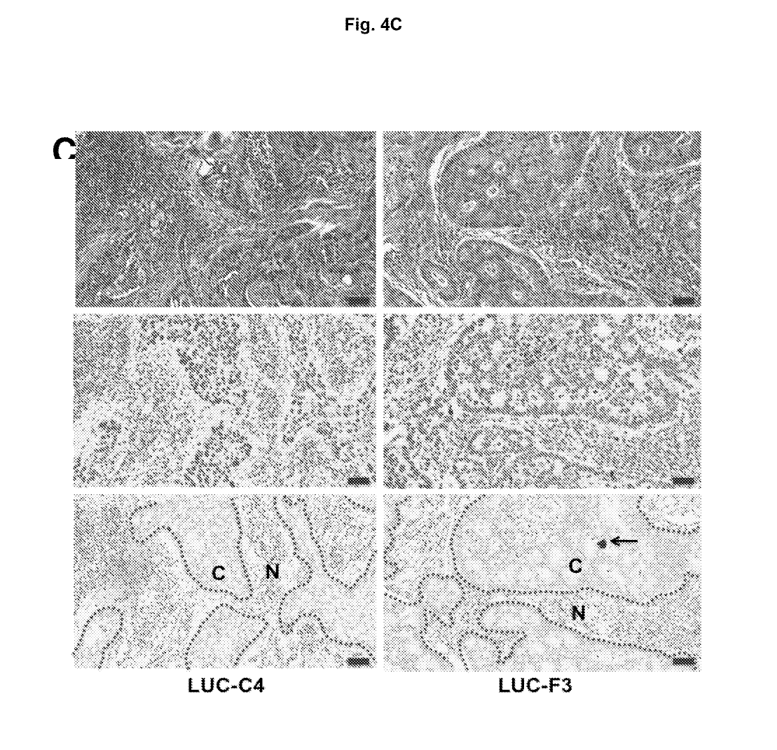

To identify a cellular source of glucose deprivation observed in the bulk tumor specimens (FIG. 1), glucose-induced uH2B levels from patient biopsies or surgery specimens were examined. As shown in FIG. 4A, breast cancer cells showed significantly less uH2B staining than their adjacent stromal cells. uH2B levels also exhibited a clear demarcation between cancer cells and their adjacent normal cells. For instance, uH2B staining was intense in both myoepithelial and luminal epithelial cells in normal breast duct (Duct 1, BC-D9 breast cancer specimen, FIG. 4A). However, uH2B levels were significantly reduced in luminal epithelial cancer cells that had undergone transformation while remained unchanged in normal luminal epithelial cells in Duct 2. In contrast, H2B levels were the same in both normal and cancer cells. In another breast cancer case, uH2B levels were also lower in cancer cells while uH2B levels remained high or unchanged in adjacent normal tissue (BC-01, FIG. 4A). Similarly, uH2B levels were also much lower in cancer cells compared to their adjacent non-cancer cells in 33 cases of additional 34 breast cancer specimens of different histopathological types, grades and stages.

To determine whether impairment of glucose-induced uH2B occurs in other types of cancer cells, tumor specimens from colon and lung cancer patients were analyzed. As shown in FIGS. 4B and 4C, impairment of uH2B was also evident in colon and lung cancer cells. Specifically, of 36 colon tumor specimens of various types, grades and stages, 31 cases showed lower uH2B levels in cancer cells compared to their stromal cells. Weak uH2B levels were also observed in 4 tumor specimens, in which no stromal cells were present. Moderate uH2B levels in one case were detected in both cancer and their stromal cells.

Of 36 lung cancer specimens of various types, grades and stages, 35 cases showed lower uH2B levels in cancer cells compared to their adjacent stromal cells. One case showed low levels of uH2B in all cells of the specimen, in which no stromal cells were present. Therefore, impairment of uH2B was detected in breast, colon and lung cancer cells compared to their stromal cells in 106 out of 109 cases. Furthermore, lower uH2B levels were also observed in cancer cells of additional 23 anatomic sites compared to their stromal tissues (n=1-3 per anatomic site, data not shown). Taken together, these results indicate that impairment of glucose-induced uH2B is characteristic of cancer cells in vivo.

Glucose is the sole nutrient inducer of uH2B in yeast and mammalian cells. uH2B levels correlated with the amounts of glucose in cultured cells by both Western blotting and immunohistochemical analyses (FIG. 2 and FIG. 3). Therefore, lower levels of uH2B in cancer cells of the tumor specimens may represent glucose deprivation in cancer cells in vivo. This is consistent with the observation that relative glucose levels were lower in bulk tumor specimens than those of normal cells of the same tissue origins (FIG. 1). uH2B levels exhibit a clear demarcation between cancer cells and their adjacent normal cells (FIG. 4). In addition, loss of uH2B occurs, to a similar extent, in all cancer cells within a cancer cell nest (FIG. 4). Taken together, these data indicate that glucose deficiency is characteristic of cancer cells in vivo.

### Example 6

### Glucose Deficiency Increases Expression of USP22

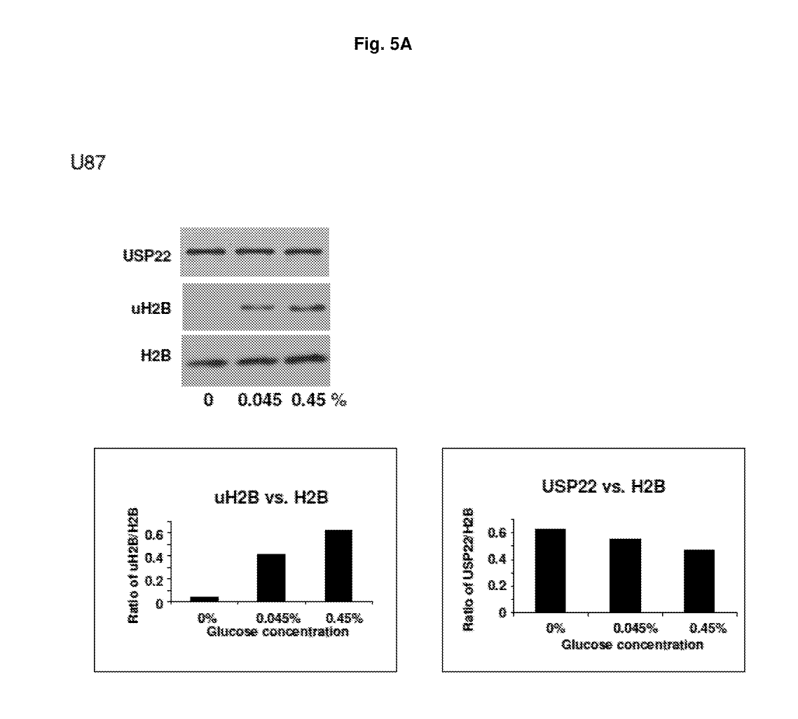

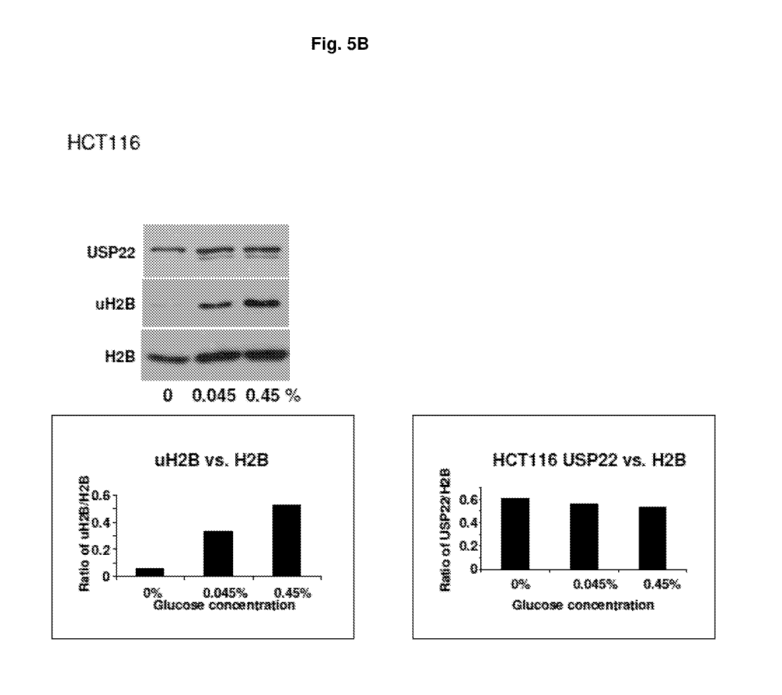

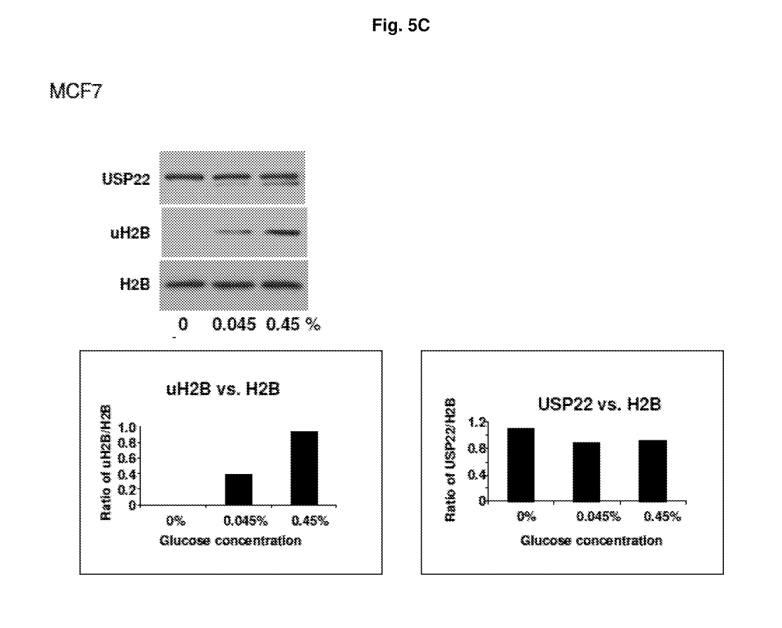

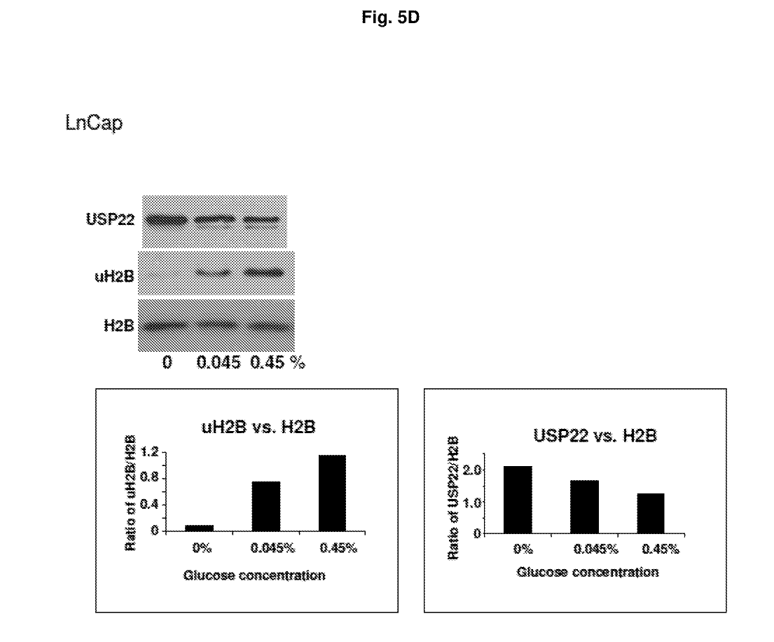

Expression of USP22 was analyzed using the same samples as described in Example 3. Expression of H2B and uH2B were also analyzed as described in Example 3. As shown in FIG. 5, expression of USP22 is induced by glucose deficiency in vitro, while expression of uH2B is inhibited.

### Example 7

### USP22 Is Up-Regulated and uH2B Is Down-Regulated in Breast, Colon, and Lung Cancer Cells of Patient Tumor Specimens

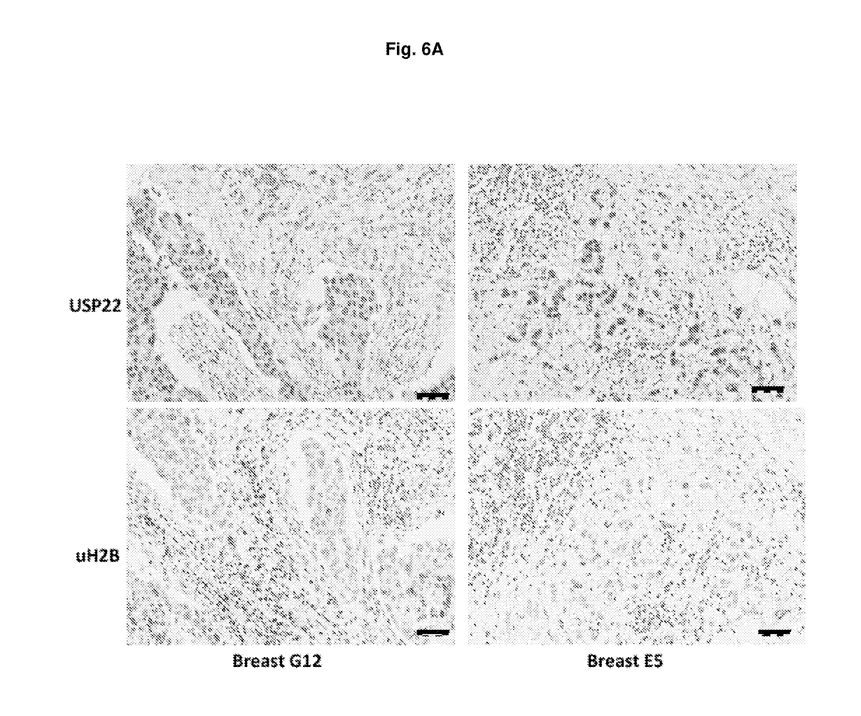

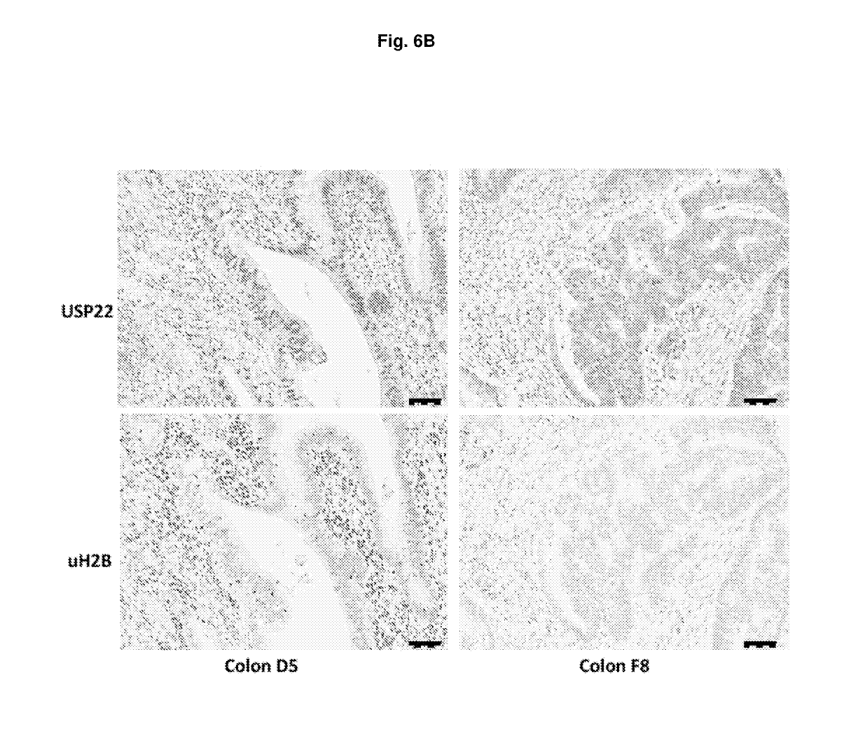

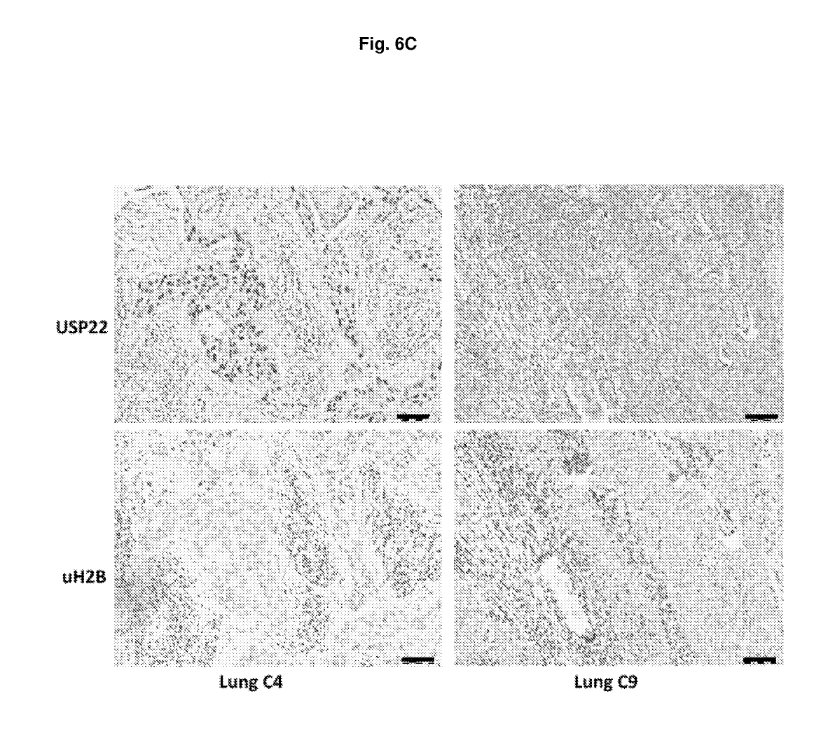

Immunohistochemistry was performed on patient tumor specimens using anti-USP22 (Sigma catalog # HPA044980) at a dilution of 1:100. Immunohistochemistry of uH2B was performed as in Example 5. As shown in FIG. 6, USP22 is up-regulated and uH2B is down-regulated in breast (FIG. 6A), colon (FIG. 6B), and lung (FIG. 6C) cancer cells from patient tumor specimens.

All references cited herein are incorporated by reference herein in their entireties.

We claim:

1. A method of treatment for cancer comprising administration to a subject in need of treatment a composition comprising a negative modulator of a deubiquitinating enzyme or a deubiquitinating enzyme cofactor in an amount effective to increase the mono-ubiquitination of a histone in cancer cells in the subject.

2. The method of claim 1 wherein the cancer is carcinoma.

3. The method of claim 1 wherein the cancer is selected from the group consisting of breast, prostate, colon, and lung cancer.

4. The method of claim 1 wherein the histone is histone H2B.

5. The method of claim 1 wherein the deubiquitinating enzyme is USP22 and the deubiquitinating enzyme cofactor is ATXN7L3, ATXN7 or ENY2.

6. The method of claim 1 wherein the negative modulator of the deubiquitinating enzyme or deubiquitinating enzyme cofactor is selected from the group consisting of: an anti- deubiquitinating enzyme or a deubiquitinating enzyme cofactor antibody, an aptamer that inhibits a deubiquitinating enzyme or a deubiquitinating enzyme cofactor, an antisense nucleic acid molecule that inhibits deubiquitinating enzyme or deubiquitinating enzyme cofactor transcription or translation, an siRNA that inhibits deubiquitinating enzyme or a deubiquitinating enzyme cofactor translation, a small molecule inhibitor of a deubiquitinating enzyme or a deubiquitinating enzyme cofactor, and combinations thereof.

7. The method of claim 1 wherein the negative modulator comprises one or more small molecule inhibitors of USP22, ATXN7L3, ATXN7 or ENY2.

8. A method of treatment for cancer comprising administration to a subject in need of treatment a composition comprising a negative modulator of glucose metabolism in an amount effective to kill cancer cells in the subject.

9. The method of claim 8 wherein the cancer is carcinoma.

10. The method of claim 8 wherein the cancer is selected from the group consisting of breast, prostate, colon, and lung cancer.

11. The method of claim 8 wherein the negative modulator of glucose metabolism is selected from the group consisting of: Phloretin, 2-deoxyglucose, 3-bromopyruvate, lonidamine, 3PO, CAP-232/TLN-232, Dichloroacetate, FX11, Oxamate, Amino oxyacetate, AZD-3965, 5-Dehydroepiandrosterone [DHEA], Oxythiamine, Tarvagenix, 6-Diazo-5-oxo-L-norleucine, 968, BPTES, GSK837149A, C75, CPI-613, Metformin, MPC-9528, disulfiram, ethylene glycol poisoning, fluoride, iodoacetate, mercury and arsenite (As₃O), sulfhydryl, and pentavalent arsenic AsO₄.

12. A method of assaying the glucose content of a cell or tissue comprising detecting the level of mono-ubiquitinated histone H2B (uH2B) in the cell or tissue.

13. The method of claim 12 wherein the level of mono-ubiquitinated histone H2B (uH2B) is detected using an anti-uH2B antibody.

14. A composition effective for the treatment of cancer comprising at least one negative modulator of a deubiquitinating enzyme or a deubiquitinating enzyme cofactor.

15. The composition of claim 14 wherein the negative modulator of the deubiquitinating enzyme or deubiquitinating enzyme cofactor is selected from the group consisting of: an anti- deubiquitinating enzyme or deubiquitinating enzyme cofactor antibody, an aptamer that inhibits deubiquitinating enzyme or deubiquitinating enzyme cofactor, an antisense nucleic acid molecule that inhibits a deubiquitinating enzyme or deubiquitinating enzyme cofactor transcription or translation, an siRNA that inhibits deubiquitinating enzyme or deubiquitinating enzyme cofactor translation, a small molecule inhibitor of deubiquitinating enzyme or deubiquitinating enzyme cofactor, and combinations thereof.

18. The composition of claim 14 wherein the deubiquitinating enzyme is USP22 and the deubiquitinating enzyme cofactor is ATXN7L3, ATXN7 or ENY2.

19. The composition of claim 14 wherein the at least one negative modulator comprises two or more different small molecule inhibitors, wherein each small molecule inhibitor is an inhibitor of USP22, ATXN7L3, ATXN7 or ENY2.
